(19) United States
(12) Patent Application Publication
(10) Pub. No.: US 2005/0009837 A1
(43) Pub. Date: Jan. 13, 2005

(54) Modulators of lipid metabolism and methods of use

(75) Inventor: Barry Forman, Irvine, CA (US)

Correspondence Address: ROTHWELL, FIGG, ERNST & MANBECK, P.C., 1425 K STREET, N.W., SUITE 800, WASHINGTON, DC, 20005, US

(73) Assignee: City of Hope, Duarte, CA (US)

(21) Appl. No.: 10/849,542

(22) Filed: May 20, 2004

Related U.S. Application Data
(60) Provisional application No. 60/471,740, filed on May 20, 2003.

(51) Int. Cl. A61K 31/496; A61K 31/433; A61K 31/427; A61K 31/422; A61K 31/4245; A61K 31/425; A61K 31/42; A61K 31/4196; A61K 31/4178

(52) U.S. Cl. 514/254.05; 514/362; 514/364; 514/365; 514/374; 514/383; 514/397; 514/372

(57) ABSTRACT

The present invention relates to methods and compositions for modulating lipogenesis, lipid accumulation and lipid metabolism in a cell. These methods and compositions modulate genes which are controlled by LXRα, including the lipogenic transcription factor SREBP-1c.

This application claims the benefit of prior co-pending U.S. Provisional Ser. No. 60/471,740 filed May 20, 2003, the disclosures of which are hereby incorporated by reference.

### BACKGROUND

1. Field of the Invention

This invention pertains to the field of medicine and particularly to methods and compounds useful for modulation of lipid metabolism.

2. Description of the Background Art

It is well known in the field of medicine that obesity and elevated serum levels of lipids and cholesterol are extremely unhealthy but common conditions. Obesity has been linked to a number of health problems, some of which are major causes of morbidity and mortality in the United States, for example, cardiovascular diseases, diabetes and cancer, as well as orthopedic problems, and, of course, social stigma.

Unfortunately, although it is clear that consumption of more calories than are burned in daily activity is an obvious cause with a seemingly simple solution, many persons remain obese despite attempts to achieve a more healthy lifestyle and the availability of largely ineffective treatments such as stimulants and catecholamine-active drugs (e.g., phenylethylamine derivatives, fenfluamine, fluoxetine). Obesity, type 2 diabetes (insulin resistance) and the “metabolic syndrome” are quite prevalent and are becoming more so.

Weight control is an essential part of treatment for such metabolic conditions as high blood cholesterol, elevated triglycerides and elevated blood sugar. Achieving reduction of weight to within the recognized normal range prevents or greatly ameliorates complications associated with these conditions, however maintaining such weight loss has proven extremely difficult for many individuals. The causes for some persons' susceptibility to increases in weight above the normal range and to elevated levels of lipids, cholesterol and blood sugar are poorly understood. Therefore, there is a great need for further understanding of the biochemical and metabolic processes which are involved in lipid and cholesterol usage and storage in the body, as well as for methods by which these processes, such as lipogenesis and lipid storage, may be modified.

Lipogenesis takes place mainly in the liver, where fatty acids are released to the blood in the form of triglycerides, and in adipocytes, where fatty acids are stored. Lipogenesis is influenced by several factors, for example it is activated after meals by elevated levels of insulin and glucose, but is repressed during fasting and by dietary polyunsaturated fatty acids (PUFAs). While fatty acids are essential as a fuel source and as membrane components, excess fatty acid formation and storage lead to obesity and associated metabolic disorders including insulin resistance, hypertension and atherosclerosis.

Nuclear receptors control different aspects of many metabolic pathways, including cholesterol and lipid metabolism. These receptors belong to a superfamily of ligand-modulated transcription factors which includes the receptors for steroid hormones, retinoids, vitamin D and thyroid hormone. In the presence of their specific ligand, nuclear hormone receptors modify transcription of specific target genes.

Nuclear receptors have conserved DNA-binding domains (DBD) that specifically bind to DNA at cis-acting elements in the promoters of their target genes. Nuclear hormone receptors also contain a C-terminal ligand binding domain (LBD) which confers ligand-specificity contains a dimerization interface, a coregulator (coactivator or corepressor) interaction domain and a transcriptional activation function. Dimerization of the nuclear receptor (homologous or heterologous) can lead to allosteric changes that promote high affinity DNA and/or ligand binding. Ligand binding results in additional conformational changes that can promote release of a corepressor and interaction with (recruitment of) a coactivator. Coactivator recruitment results in a receptor complex which has a high affinity for a specific DNA region and which can modulate the transcription of one or more specific genes. Because the coactivator can modify chromatin structure and basal transcriptional machinery, ligand binding to the LBD activates transcription.

A nuclear receptor antagonist is a ligand that either represses or fails to activate the receptor upon binding. An antagonist can function by recruiting copressor or by failing to recruit coactivator. In the latter case, the antagonist is an inactive competitor that competes for binding with an agonist. As will be apparent to those skilled in the art, an agonist of a receptor that effects negative transcriptional control over a particular gene will actually decrease expression of the gene. Conversely, an antagonist of such a receptor will increase expression of the gene.

Mevalonic acid and oxysterols are known to affect LXRα activity. These findings suggested that LXRα may represent a component of the signaling pathway which controls cholesterol metabolism, including the gene encoding the enzyme Cyp7A. This enzyme is responsible for the rate-limiting step in conversion of cholesterol to bile acids, a necessary step for removal of cholesterol from the body.

Lipogenesis is regulated mainly at the transcriptional level. The basic helix-loop-helix leucine zipper transcription factor sterol regulatory element binding protein-1c (SREBP-1c, also called ADD-1) is very important in lipid metabolism and lipogenesis in particular. SREBP-1c belongs to a family comprising SREBP-1 and SREBP-2, each of which is encoded by a different gene. Horton et al., J. Clin. Invest. 109:1125-1131, 2002. SREBP-2 regulates the expression of genes involved in cholesterol synthesis while SREBP-1 regulates lipogenic genes. The SREBP-1 gene generates at least two isoforms, SREBP-1a and SREBP-1c, which differ in their first exon. SREBP-1c, although a weaker transcriptional activator than SREBP-1a, is expressed more abundantly than SREBP-1a in the liver and adipose tissue and is the main activator of lipogenesis in vivo. Shimano et al., J. Clin. Invest. 99:846-854, 1997; Shimomura et al., J. Clin. Invest. 99:836-845, 1997. SREBP-1c directly activates the transcription of genes involved in the biosynthesis of fatty acids such as acetyl-CoA carboxylase (ACC), fatty acid synthase (FAS) and stearoyl-CoA desaturase (SCD-1) in response to lipogenic signals; genetic manipulations of this gene in cell lines and in mice have demonstrated the importance of SREBP-1c in lipogenesis. Horton et al., J. Clin. Invest. 109:1125-1131, 2002.

LXR is an important cholesterol and fatty acid regulator. LXRα and LXRβ are encoded by two different genes, but share a number of characteristics. For example, they share extensive amino acid identity and they recognize the same or similar response elements. LXRα and LXRβ are activated by natural ligands, mainly oxidized cholesterol metabolites, and by synthetic agonists. Janowski et al., Nature 383:728-731, 1996; Lehmann et al., J. Biol. Chem. 272:3137-3140, 1997; Schultz et al., Genes Dev. 14:2831-2838, 2000. Notably, both LXRα and LXRβ can exhibit some constitutive activity in the absence of ligand. Forman et al., Proc. Natl. Acad. Sci USA 94:10588-10593, 1997; Apfel et al., Mol. Cell. Biol. 14:7025-7035, 1994; Song et al., Proc. Natl. Acad. Sci. USA 91: 10809-10813, 1994; Teboul et al., Proc. Natl. Acad. Sci. USA 92:2096-2100, 1995. The constitutive activity appears to reflect a combination of ligand-independent activity and activation by an endogenous mevalonic acid-derived metabolite.

The LXRs also differ in several ways. LXRα is expressed in a restricted number of tissues including liver, intestine, fat, macrophages and adrenal glands and plays an important role in regulating lipid homeostasis. Apfel et al., Mol. Cell. Biol. 14:7025-7035, 1994; Willy et al. Genes Dev. 9:1033-1045, 1995; Schultz et al., Genes Dev. 14:2831-2838, 2000; Peet al., Cell 93:693-704, 1998. On the other hand, LXRβ is expressed ubiquitously and may be involved in lipid homeostasis (Juret al., Mol. Endocrinol. 17(2):172-182, 2003) and in certain diseases such as Alzheimer's disease and other neurological disorders. Whitney et al., Mol. Endocrinol. 16(6): 1378-1385, 2002; Wang et al., Proc. Natl. Acad. Sci. USA 99(21): 13878-13883, 2002; Fukumoto et al., J. Biol. Chem. 277(50): 48508-48513, 2002; Koldamova et al., J. Biol. Chem., 2003; Song et al., Proc. Natl. Acad. Sci. USA 91:10809-10813, 1994; Teboul et al., Proc. Natl. Acad. Sci. USA 92:2096-2100, 1995; Seol et al., Mol. Endocrinol. 9:72-85, 1995.

The first piece of evidence involving LXRα in cholesterol homeostasis came from the observation that LXRα null mice accumulate large amounts of sterol in their liver when fed a diet containing 2% cholesterol. Peet et al., Cell 93:693-704, 1998. Since then, numerous further reports have supported a role for LXRα in cholesterol homeostasis in several tissues. For example, in macrophages LXRα activation promotes cholesterol efflux by inducing the ATP-binding cassette transporters (ABCA1) and by inducing apolipoprotein E (ApOE) and ABCG5 and ABCG8. Repa et al., Science 289:1524-1529, 2000; Costet et al., J. Biol. Chem. 275:28240-28245, 2000; Laffitte et al., Proc. Natl. Acad. Sci. USA 98:507-512, 2001. The importance of ABCA1, ABCG5 and ABCG8 in cholesterol efflux and of ApoE as a protective factor against atherosclerosis is well documented. See Repa and Mangelsdorf, Nat. Med. 8(11):1243-1248, 2002. ABCA1 has been identified as responsible for a rate-limiting step in the efflux of cholesterol from peripheral cells. ABCG5 and ABCG8 are LXR target genes which may be responsible for LXR-mediated increases in cholesterol hepatobiliary secretion. LXRα therefore may be a protective factor in the development of atherosclerosis by regulating these genes in various tissues, including atherosclerotic plaque resident macrophages (foam cells), intestine and liver. In the intestine, LXRα may prevent cholesterol absorption by regulating ABCA1, ABCG5 and ABCG8. Yu et al., J. Biol. Chem., 2003; Repa et al., J. Biol. Chem. 277(21):18793-18800, 2002; Repa et al., Science 289:1524-1529, 2000.

Further, risk for development of Alzheimer's Disease (AD) is associated with high serum cholesterol. LXR activation induces ABCA1, which in turn leads to increased secretion of Aβ peptide. Therefore LXR agonists may increase deposition of Aβ peptide, as seen in AD. See Fukumoto et al., J. Biol. Chem. 277(50):48508-48513, 2002.

LXRα also is involved in lipogenesis in the central nervous system as well as other tissues. Mice fed a specific LXR agonist, T0901317, have elevated plasma triglycerides compared to mice fed a control diet. Schultz et al., Genes Dev. 14:2831-2838, 2000. LXRα null mice are deficient in genes involved in lipogenesis, including SREBP-1c and its downstream targets, ACC and FAS. Repa et al., Genes Dev. 14:2819-2830, 2000. Although the regulation of lipogenic genes by LXRα is attributed mainly to its ability to regulate SREBP-1c, LXRα also can regulate some SREBP-1c target genes independently. Repa et al., Genes Dev. 14:2819-2830, 2000; Yoshikawa et al., Mol. Cell. Biol. 21:2991-3000, 2001. For example, the FAS promoter contains response elements for both SREBP-1c and LXR. Joseph et al., J. Biol. Chem. 277:11019-11025, 2002. Thus, while LXRα activation may lower cholesterol levels, it may concomitantly elevate triglycerides to undesirable levels. Ou et al. found that PUFAs bind LXR and repress its activity. Ou et al., Proc. Natl. Acad. Sci. USA 98:6027-6032, 2001. PUFAs may have other effects on LXR. Yoshikawa et al. found that some PUFAs can repress LXR binding to DNA. Yoshikawa et al., J. Biol. Chem. 277:1705-1711, 2002.

A better understanding of how LXRα is involved in lipid and cholesterol metabolism and new methods for modifying the effects of LXRα on this metabolism would be of great use in providing methods to modify lipid use in the body, including de novo cholesterol synthesis, cholesterol catabolism, lipogenesis, lipid storage and other metabolic pathways connected to common metabolic disorders such as obesity and the metabolic syndrome, as well as related syndromes, including atheroscleosis and type 2 diabetes. Methods of modifying lipid metabolism would be of great benefit to patients suffering from conditions involving or caused by increased lipogenesis or lipid storage and high serum cholesterol or other syndromes associated with increased cholesterol, for example AD.

### SUMMARY OF THE INVENTION

In one embodiment, the invention provides methods of modulating LXR activity and methods of modulating lipid accumulation and metabolism which involve contacting cells with compounds that modulate LXR activity, for example compounds that have been identified by in vitro screening methods. In a further embodiment, the invention provides methods of modifying lipid metabolism by administering an LXR ligand, including LXR agonists and antagonists.

Compounds of the formula 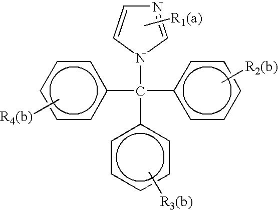
are provided.

Other embodiments of the invention, in accordance with the scope of the claims, will be appreciated by those skilled in the art.

### BRIEF DESCRIPTION OF THE DRAWINGS

FIG. 1 shows inhibition of of hLXRα activity by 20 μM Clotrimazole, S883417 and S100250.

FIG. 2 shows inhibition of LXR ligand binding domain mediated activity (reporter gene UASGx4 TK-Luc) by 20 μM Clotrimazole, S883417 and S100250.

FIG. 3 shows inhibition of LXR-coactivator association by 20 μM Clotrimazole, S883417 and S100250.

FIG. 4 is a bar graph that shows reporter gene activity in transiently transfected cells expressing the indicated ligand binding domains after contact with Clotrimazole or control.

FIG. 5 is a Clotrimazole dose-response curve for repression of LXR activity.

FIG. 6 is a bar graph presenting data showing displacement of coactivator by the indicated ligands in a mammalian two-hybrid assay.

FIG. 7 is a polyacrylamide gel presenting results of a coactivator recruitment displacement assay for recruitment of SRC-1 in the presence and absence of Clotrimazole.

FIG. 8 shows SREBP-1c promoter activity in the presence of no LXR, LXRα or LXRβ, with and without Clotrimazole.

FIG. 9 shows sterol response element (SRE) reporter activity in cells treated with Clotrimazole, 25-hydroxycholesterol or control.

FIG. 10 presents real-time PCR results for SREBP-1c expression and an SREBP-1c target gene, ACCα type II, under the indicated drug treatment conditions.

FIG. 11 is a photograph of NIH3T3-L1 adipocytes showing lipid content under the indicated drug treatment conditions.

### DETAILED DESCRIPTION OF THE INVENTION

This invention provides a method for modulating the transcription of genes regulated by the LXRα or LXRβ receptor and hence a method of inhibiting or activating cellular lipogenesis and lipid accumulation in cells. The invention also provides compounds that modulate LXRα-controlled gene expression.

LXRα or LXRβ plays a central role in fatty acid synthesis by regulating the lipogenic transcription factor SREBP-1c. Since elevated fatty acid production is linked to common disorders such as obesity and insulin resistance, LXRα inhibitors represent interesting drugs for controlling these diseases. In addition, an LXR antagonist was identified from liver by gas chromatography and mass spectrometry as the polyunsaturated fatty acid (PUFA) αlinoleic acid. Several PUFAs, unlike saturated fatty acids, repress LXRα activity and are ligands for LXRα, as well as the anti-fungal drug Clotrimazole. Clotrimazole repressed the expression of the LXR target gene SREBP-1c (as well as SREBP-1c downstream targets), resulting in repressed fatty acid formation. These findings have identified new pathways that can be manipulated pharmacologically to regulate the levels of fatty acid production. Taken together with Clotrimazole's repression of LXR activity, the present data show that it is possible to pharmacologically manipulate the activity of LXR to control lipogenesis.

Repression of LXR by Clotrimazole leads to repression of fatty acid formation by inhibiting SREBP-1c and its downstream targets. Therefore, lipogenesis can be inhibited by inhibiting LXR activity. These results provide a “proof of principle” that LXR antagonists repressors can serve as anti-lipogenic agents. Although Clotrimazole is a strong LXR antagonist, it is not specific for this receptor. Indeed, Clotrimazole also can bind other nuclear receptors. Clotrimazole acts as an agonist on mouse and human PXR and antagonizes human CAR. The effects of Clotrimazole here are not likely to be due to its ability to regulate these or any other nuclear receptors, because there is no evidence that PXR or CAR affect cholesterol or fatty acid metabolism and these receptors are not expressed in 3T3-U adipocytes. Importantly, Clotrimazole had no effect on PPARα or RXR activity (data not shown), two nuclear receptors which do have important roles in lipid homeostasis.

LXR exhibits some constitutive activity (especially when assayed in media depleted of fatty acids). This constitutive activity allows one to screen for LXR antagonists without having to preactivate the receptor. Since serum contains numerous small lipophilic molecules that regulate the activity of several nuclear receptors, LXR activity was compared in standard and lipid-stripped serum. To eliminate the effect of ligands on endogenous receptors from the assay, Gal4 DNA binding domain (DBD) and LXRα ligand binding domain (LBD) fusion proteins served as receptor.

NIH 3T3-L1 cells were maintained in growth media consisting of Dulbecco's modified Eagle's medium (DMEM) supplemented with 10% calf bovine serum (CBS), 50 U/ml penicillin G and 50 μg/ml streptomycin sulfate. To induce NIH 3T3-L1 differentiation into adipocytes, confluent cells were transferred from their growth media to DMEM containing 10% fetal bovine serum (FBS), 5 μg/ml insulin, 0.5 mM 3-isobutyl-1-methylxanthine (IBMX) and 1 μM dexamethasone for 7 days. During the differentiation period, cells also optionally received 200 nM T0901317, 7.5 μM Clotrimazole or both. No toxicity was observed with any of these compounds at the doses used.

Coactivator recruitment assays have become established as a reliable method to identify and test the activity of orphan receptor ligands (Blumberg et al., Genes Dev. 12:1269-1277, 1998; Forman et al., Nature 395:612-615, 1998; Kliewer et al., Cell 92:73-82, 1998; Krey et al., Mol. Endocrinol. 11:779-791, 1997. Therefore, mammalian two-hybrid in vitro coactivator recruitment assays were used to examine whether putative ligands could promote a functional association between LXRα or LXRβ and a coactivator as a test of a ligand's ability to modify the transcription of genes regulated by LXR. The coactivator recruitment assay efficiently detected compounds which were able to promote a functional interaction between LXR and coactivator.

In vitro coactivator recruitment assays are performed by adding the ligand to a mixture of the following components: LXRα or LXRβ Sa RXR, a coactivator and labeled LXR response element (for use as a probe). A peptide containing the receptor interaction domains of a co-activator such as GRIP1 may be used as the coactivator. SRC-1 may be expressed in bacteria and purified for these assays using the GST-GRIP1 construct, containing the three receptor interaction domains of mouse SRC-1, fused to glutathione-S-transferase. Other suitable coactivators are known in the art, for example PBP/DRIP 205/TRAP 220, SRC-1, ACTR, ASC2, PGC-1 and may be used with the inventive methods disclosed here. Any functional coactivator or coactivator complex is contemplated.

Response elements suitable for use in this assay may be any nucleic acid probe which is substantially homologous to the target DNA sequence of the LXR being assayed and which is compatible with the assay system. Substantially homologous sequences (probes) are sequences which bind the receptor under the conditions of the assay. Response elements can be modified by methods known in the art to increase or decrease the binding of the response element to the nuclear receptor. The response element used in the specific assays exemplified here was an LXRE from the SREBP-1C promoter (nt 337-361, accession no. ABO46200).

Transcription of an LXR target gene such as CYP7A (rodents only), ABCA1, ABCG5/ABCG8, SR-B, SREBP-1C, CPC, PIPOE, aPoc11, PCTP, SHP (human), TNFα or any LXR target gene can be modulated by administering an LXR ligand (agonist or antagonist) compound to a cell which expresses the LXR receptor. Exogenous LXR ligands may be used to modify the regulation of ABCA1, ABCG5, ABCG8, SREBP-1c or any other LXR target gene. Manipulation of LXR with receptor-binding ligands thus provides a treatment for hypercholesterolemia, elevated triglyceride levels and any other metabolic process disorder or syndrome which may be controlled or ameliorated through LXR, including AD. Preferred ligands may be derivatives of natural compounds, synthetic or semi-synthetic molecules. For example, Clotrimazole, S883417 and S100250 all have been found to be LXR ligands. Compounds which bind to and which are useful for modulating LXR include compounds of the formula 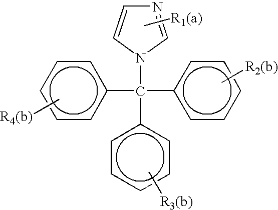
wherein (a) represents an integer from 0 to 3 and each (b) may be the same or different and represents an integer from 0 to 5;
wherein each R₁, R₂, R₃ and R₄ may be the same as or different from any other R₁, R₂, R₃ or R₄ and represents a moiety selected from the group consisting of C₁₋₄ alkyl, C₁₋₄ alkenyl, aryl, alkylaryl, halo, trihalomethyl, furanyl, thiophenyl, pyrrolyl, pyrazolyl, diazolyl, triazolyl, tetrazolyl, dithiolyl, oxathiolyl, oxazolyl, isoxazolyl, thiazolyl, isothiazolyl, oxadiazolyl, oxatriazolyl, dioxazolyl, isoxazinyl and piperazinyl, wherein said moiety may be unsubstituted or substituted with one or more substituent selected from the group consisting from methyl, ethyl, amino, halo, trihalomethyl and nitro. These compounds also are useful as lead compounds for the discovery of new compounds in screening assays. Compounds according to Formula I described above are suitable compounds for use in the methods of this invention. In particular, Clotrimazole, S883417 and S100250 have been found to modulate the transcription of endogenous genes or reporter genes controlled by LXR.

The invention is further described and illustrated in the following examples, which are not intended to be limiting.

### EXAMPLES

### Example 1

### Selection of LXRα

### Receptor Inhibiting Compounds

CV-1 cells were grown in DMEM supplemented with 10% resin-charcoal stripped fetal bovine serum, 50 U/ml penicillin G and 50 μg/ml streptomycin sulfate at 37° C. in 5% CO₂. Cells were plated to 50-80% confluence one day prior to transfection using phenol red-free DMEM-FBS. The cells were transfected by lipofection using N-[1-(2,3-dioleoyloxy)propyl]-N,N,N-ammonium methyl sulfate according to manufacturer's instructions (Boehringer Mannheim).

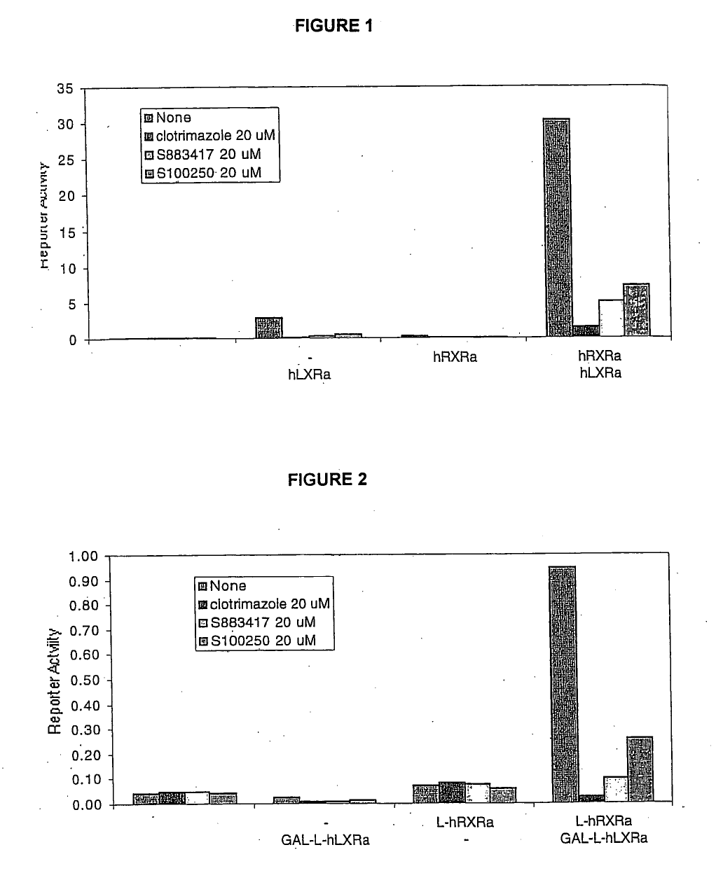

Genes encoding the following full-length proteins, which are suitable for use in the studies described herein, were cloned into pCMX: human RXRα (Gene Bank accession X52773) and human LXRα (GenBank accession U22662). The cells were transfected with CMX-βgal and luciferase constructs. The CMX-βGal construct contained the ligand binding domain of human RXR (hRXRα) and the ligand binding domain of human LXRα (hLXRα) as indicated in FIG. 1. The luciferase reporter construct (LXREx3 TK-Luc) contained the herpes virus thymidine kinase promoter (−105/+51) linked to three copies of the response element LXRE. After transfection, the cells were treated with 20 μM of a synthetic azole test compound or vehicle alone for 40 hours in phenol red-free DMEM-FBS. The structures of the azole compounds used (Clotrimazole, S883417 and S100250) are provided in Table II, below. After exposure to the candidate antagonist compounds, cells were harvested and assayed for luciferase and β-galactosidase activity. Results are provided in FIG. 1. The candidate azole compound ketonazole also was tested, but found to be inactive (data not shown), while all three of the other azole compounds were able to antagonize the constitutive activation mediated by LXR.

TABLE I
Chemical Structures of Azole Antagonists of LXRα.

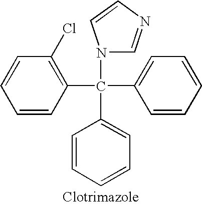

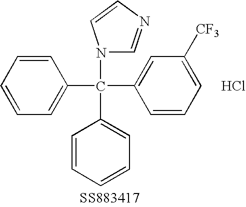

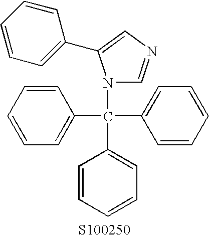

### Example 2

### Inhibition of LXR Binding Domain by Azole Compounds

The above example was repeated, except transfection was performed with the genes indicated in FIG. 2. The reporter gene was UASGx4 TK-Luc. The results, presented in FIG. 2, show that Clotrimazole, S883417 and S100250 are able to inhibit constitutive activation by the ligand binding domain of LXRα-RXR heterodimers.

### Example 3

### Inhibition of LXR-Coactivator Association

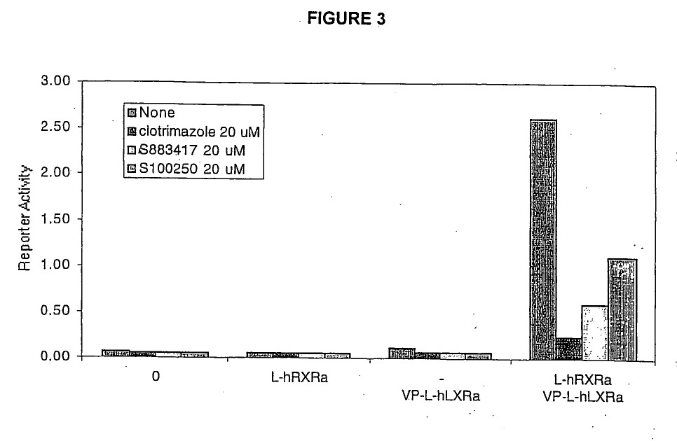

The method of Example 1 was repeated, except the cells were transfected with constructs containing the genes indicated in FIG. 3. VP-L-hLXRα contains the transactivation domain of Herpes VP16, linked to the LBD of human LXRα, L-hRXRα is the LBD of human RXRα. UASGx4 TK-Luc was the reporter gene, and the assay additionally included the coactivator GAL4-SRC1. Treatment with 20 μM azole compound as indicated was able to inhibit interactions between LXRα-RXR heterodimers and the receptor interaction domains from the coactivator SRC1. See FIG. 3.

### Example 4

### Screening Method for Specific LXR Inhibitors

Synthetic compounds were screened in an effort to find more specific LXR inhibitors. CV-1 cells were maintained and transfected using known methods. Forman et al., Cell 81: 687-693, 1995. CV-1 cells were transiently transfected with Gal-hLXRα, the RXR LBD, the UAS_{G}x4 TK Luc reporter and CMX-β-gal as an internal control, either in standard or in lipid-stripped serum. After transfection, cells were treated for approximately 40 hours with phenol red-free DMEM-FBS containing the indicated compounds at the following concentrations: T0901317 (100 nM), Clotrimazole (10 μM), S883417(10 μM) and S100250 (10 μM), unless otherwise specified. After exposure to ligand, the cells were harvested and assayed for luciferase and β-galactosidase activity. The luciferase activity was normalized to the internal β-galactosidase control (reporter activity). All points were assayed in triplicate and varied by less than 15%. Each experiment was repeated three or more times with similar results. No cytotoxicity was observed.

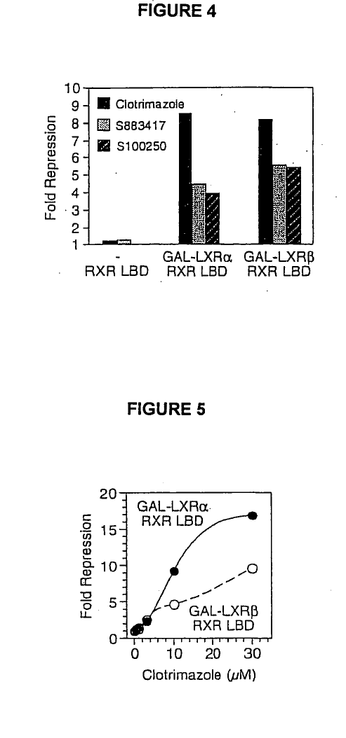

In the transient transfection assays, three related compounds (see Table I) repressed LXR activity. The ability of these compounds to repress LXRα and LXRβ was compared by transfecting CV-1 cells with either the RXR LBD alone or the RXR LBD together with either Gal-LXRα or Gal-LXRβ. In this case, transfections were performed in lipid-stripped serum as above, but treated with the ligands indicated in FIG. 4. Clotrimazole was a powerful antagonist of both LXRα and LXRβ, but had no effect on RXR. See FIG. 4. The Clotrimazole derivatives S883417 and S100250 also repressed both LXRs, but were approximately 50% less effective than Clotrimazole.

Dose-response analysis showed that Clotrimazole repressed both LXRα and LXRβ constitutive activity in a dose-dependent manner with an EC₅₀ value of approximately 10 μM. See FIG. 5. Transfections were performed as described above, with increasing concentrations of Clotrimazole. As with PUFAs (data not shown), Clotrimazole was a more potent antagonist of LXRα than LXRβ. See FIG. 5.

### Example 5

### Coactivator Displacement by Identified LXR Antagonists

One characteristic of nuclear receptor antagonists is their ability to displace coactivators bound to ligand-activated receptors. Thus, if Clotrimazole is an antagonist of LXR, it should inhibit the recruitment of coactivators. To test this, a mammalian two-hybrid assay was used. In this assay, CV-1 cells were transfected with Gal-SRC-1, VP-LXRα or VP-LXRβ, the RXR LBD and a reporter construct. In this type of assay, therefore, only a protein-protein interaction between the coactivator SRC-1 and LXR can result in transcriptional activity.

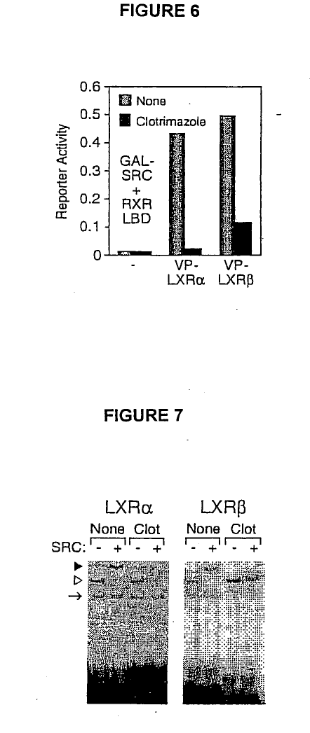

The mammalian two-hybrid assay of FIG. 6 was performed by transfecting CV-1 cells with expression plasmids for Gal-SRC-1, VP-LXRα LBD or VP-LXRβ LBD, the RXR LBD and the USA_{G}x4 Tkluc reporter. Cells were treated for 40 hours with Clotrimazole before luciferase and β-galactosidase activities were measured. Gal-SRC in FIG. 6 refers to a fusion between the Gal4 DNA binding domain and the three receptor interaction domains of SRC-1.

As expected from constitutively active receptors, both LXRα and LXRβ interacted strongly with the coactivator SRC-1 in the absence of ligand. See FIG. 6. The addition of Clotrimazole inhibited this interaction. This was a specific effect because Clotrimazole did not inhibit basal transcription.

This was tested more directly using an in vitro coactivator recruitment/coactivator displacement assay. See FIG. 7. This assay has the advantage that it is performed with receptors bound to DNA and that it measures a direct interaction between receptor and coactivator with no interference by other proteins that may indirectly stabilize the complex. The assay was performed by mixing in vitro translated LXRα or LXRβ, RXR, 5 μg of purified GST-SRC-1 and a ³²P-labeled LXRE probe with or without 10 μM Clotrimazole according to previously described methods. Wang et al., Mol. Cell 3:543-553, 1999. The complexes then were separated through 5.5% polyacrylamide gels. The probe used represents a DR-4 element derived from the human SREBP-1c promoter (nt 337-361, accession no. AB046200).

As expected, LXR-RXR heterodimers recruited SRC-1 in the absence of ligand (FIG. 7, lanes 2 and 6). The addition of Clotrimazole had no effect on the LXR-RXR heterodimers (lanes 3 and 7), but prevented the interaction with SRC-1 (lanes 4 and 8). These results further confirm that Clotrimazole is an antagonist ligand for LXR.

### Example 6

### Antagonism of SREBP-1c Promoter by Clotrimazole

To test whether Clotrimazole exhibits antagonist activities in a relevant context related to lipogenesis in cells, it was necessary to study its effects on the SREBP-1c promoter. This promoter is directly regulated by LXR via two LXREs present within the first 240 base pairs of the proximal promoter. Thus, we transfected CV-1 cells with the −241/+12 mSREBP-1c promoter along with LXRα or LXRβ. CV-1 cells were transiently transfected with full length LXRα or LXRβ, the SREBP-1c promoter or the SREBP-1c LXREm reporter and CMX-β-gal (as an internal control) in lipid-stripped serum. After transfection, the cells were treated with 10 μM Clotrimazole. The luciferase activity was normalized to the internal β-galactosidase control (reporter activity).

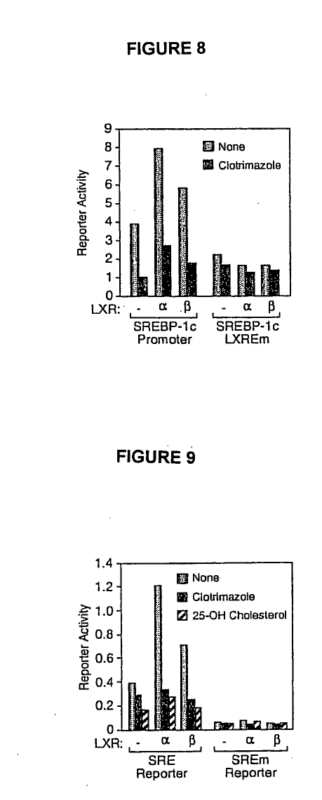

The SREBP-1c promoter was active even in the absence of LXR and the activity was repressed by Clotrimazole. See FIG. 8. The basal activity of this reporter was increased by co-transfecting with either LXRα or LXβ. In each case, Clotrimazole repressed the LXR-induced activity. When cells were transfected with an SREBP-1c promoter mutant where both LXREs were inactivated (SREBP-1c LXREm), the basal activity of this mutant was almost 50% lower than that of the wild-type promoter. This confirms that these results were dependent on LXR and is consistent with these cells containing endogenous LXR. More importantly, expression of either LXRα or LXRβ had no stimulatory effect on this reporter construct. Furthermore, Clotrimazole did not repress the promoter activity in the absence or the presence of LXR. See FIG. 8. Together, these results demonstrate that LXR activates a relevant target gene in the absence of exogenously added ligand and that Clotrimazole's antagonism of LXR is dependent on the presence of LXREs.

### Example 7

### LXR Regulation of Activities Downstream to SREBP-1c

Since LXR regulates SREBP-1c expression, LXR also should regulate activities downstream of SREBP-1c. To test this, CV-1 cells were transiently transfected with as in Example 6, but with the SRE or the SREm reporter, SREBP-1c-dependent reporter constructs consisting of the FAS proximal promoter linked to the luciferase gene. This reporter does not contain any LXRE and is therefore not regulated directly by LXR (our unpublished observations), but contains two binding sites for SREBP-1c (one SRE and one E-box). See Wang et al., Mol. Cell 3:543-553, 1999, the disclosures of which are hereby incorporated by reference. Cells then were treated as in Example 6 with the indicated compounds, Clotrimazole or 25-hydroxycholesterol (25-OH cholesterol). 25-OH cholesterol is known to repress the synthesis of SREBP-1c and is therefore used to eliminate the effects of this transcription factor on the FAS promoter (SRE). In the absence of LXR, the SRE reporter displayed some activity, presumably due to the presence of endogenous activators. See FIG. 9. Clotrimazole had only a weak repressive effect while 25-OH cholesterol repressed the reporter by 50%. These results indicate that SREBP-1c, but not LXR, is responsible for some of the constitutive activity of this reporter.

When LXRα was transfected along with the SRE reporter, there was a 3-fold increase in activity; LXRβ co-expression resulted in almost a 2-fold increase. See FIG. 9. The addition of Clotrimazole or 25-OH cholesterol prevented this induction. That Clotrimazole and 25-OH cholesterol have the same effect on this reporter indicates that they regulate a common pathway, namely that they both inhibit SREBP-1c. To confirm that the reporter activity observed by transfecting LXR or by adding Clotrimazole is dependent on SREBP-1c, the SREBP-1c response elements in the reporter construct were mutated. The basal activity of this reporter was decreased significantly (FIG. 9), further supporting the fact that SREBP-1c is required to induce this reporter. Secondly, neither LXRα nor LXRβ were able to activate the reporter containing the mutated SRE and Clotrimazole and 25-OH cholesterol had no significant effects on the already very low activity of this reporter. See FIG. 9. These results demonstrate that LXR regulates this promoter via SREBP-1c. These results also indicate that LXR and its ligands regulate, in a predictable manner, SREBP-1c expression as well as SREBP-1c downstream targets. This has important implications since SREBP-1c is a crucial regulator of lipogenesis.

### Example 8

### Direct Assay of SREBP-1c Downregulation by Clotrimazole

To test directly whether Clotrimazole can downregulate SREBP-1c, real-time PCR was performed on cDNA derived from HepG2 cells treated with a known LXR agonist ligand, T0901317 (100 nM), or with T0901317 (100 nM) plus Clotrimazole. (10 μm) for 24 hours. Real time PCR was then performed on cDNA derived from RNA isolated form the treated HepG2 cells as follows.

Real time quantitative PCR assays were performed using an Applied Biosystems 7700 sequence detector (Applied Biosystems). Each reaction mixture contained 50 ng cDNA, 1.25 pmol each forward and reverse primer, and 12.5 l 2× SYBR Green PCR master mix (Applied Biosystems) in a final volume of 25 μl. The PCR conditions were 50° C. for 2 min, 95° C. for 10 min, and 40 cycles of 95° C. for 15 sec and 60° C. for 1 min. Samples were run in duplicate for each primer pair. Expression of glyceraldehyde-3-phosphate dehydrogenase (GAPDH) was analyzed in parallel as an internal control. Each sample was normalized to GAPDH and is represented in the Figure showing the results as arbitrary units with the expression levels in untreated cells assigned a value of one. The primer sequences are as follows: human SREBP-1c forward 5′-CGGAGCCATGGATTGCACTTT-3′ (SEQ ID NO:1); human SREBP-1c reverse 5′-CTCAATGTGGCAGGAGGTGGA-3′ (SEQ ID NO:2); human GAPDH forward 5′-ACGCATTTGGTCGTATTGGG-3′ (SEQ ID NO:3) and human GAPDH reverse 5′-TGATTTTGGAGGGATCTCGC-3′ (SEQ ID NO:4). The primers for SREBP-1c are specific and do not recognize SREBP-1a(3).

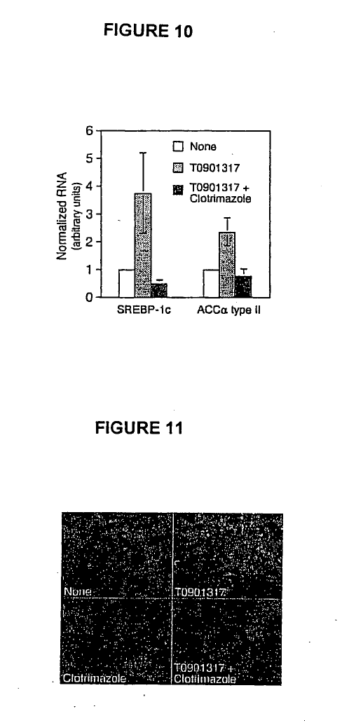

T0901317 increased the expression of SREBP-1c up to 4-fold. See FIG. 10. This is consistent with results of previous reports. See Schultz et al., Genes Dev. 14:2831-2838, 1997; Debose-Boyd et al., Proc. Natl. Acad. Sci. USA 98:1477-1482, 2001. Because SREBP-1c expression is very low in HepG2 cells it was difficult to assess repression of basal levels. Therefore, the ability to Clotrimazole to repress the T0901317-dependent elevated expression of SREBP-1c was examined. In HepG2 cells treated with both T0901317 and Clotrimazole, SREBP-1c expression was significantly reduced compared to cells treated with T0901317 alone. See FIG. 10. A similar pattern of regulation of T0901317 and Clotrimazole exists for ACCα type II, an SREBP-1c target gene. See Lopez et al., Proc. Natl. Acad. Sci. USA 93:1049-1053, 1996.

### Example 9

### LXR Ligands Modulate Cellular Fatty Acid Accumulation

LXR and its ligands regulate SREBP-1c and its downstream targets, therefore these ligands should regulate lipogenesis. To show regulation of lipogenesis in a well-recognized cellular model, NIH3T3-L1 cells were differentiated to adipocytes and tested for the presence and absence of LXR ligands for fatty acid accumulation.

NIH3T3-L1 cells were allowed to differentiate for 7 days in the presence of a well-established differentiation cocktail containing dexamethazole (1 μm), IBMX (0.5 mM) and insulin (5 μg/ml) in the absence or presence of T0901317 (200 nM) or Clotrimazole (7.5 μm). The cells then were fixed and stained with Nile Red, which stains triglycerides (TG) green, and DAPI to visualize nuclei (in blue). TG content of the cell reflected increased or decreased lipogenesis. Addition of T0901317 to the differentiation cocktail resulted in a small overall increase in TG content. See FIG. 11. Clotrimazole had the opposite effect: TG content was decreased compared to cells receiving only the differentiation cocktail. The addition of both ligands resulted in a reduction in TG content compared to treatment with T0901317 alone. See FIG. 11. These results confirm that LXR and its ligands regulate lipogenesis in cells by regulating SREBP-1c and its downstream targets.

### REFERENCES

The following references each are hereby incorporated by reference in their entirety.
1. Horton et al., J. Clin. Invest. 109:1125-1131, 2002.
2. Shimano et al., J. Clin. Invest. 99:846-854, 1997.
3. Shimomura, et al., J. Clin. Invest. 99:838-845, 1997.
4. Janowski, et al., Nature 383:728-731, 1996.
5. Lehmann, et al., J. Biol. Chem. 272:3137-3140, 1997.
6. Schultz, et al., Genes Dev. 14:2831-2838, 1997.
7. Forman, et al., Proc. Natl. Acad. Sci. USA 94:10588-10593, 1997.
8. Apfel, et al., Mol. Cell. Biol. 14:7025-7035, 1994.
9. Song, et al., Proc. Natl. Acad. Sci. USA 91:10809-10813, 1994.
10. Teboul, et al., Proc. Natl. Acad. Sci. USA 92:2096-2100, 1995.
11. Willy, et al., Genes Dev. 9:1033-1045, 1995.
12. Peet, et al., Cell 93:693-704, 1998.
13. Seol et al., Mol. Endocrinol. 9:72-85, 1995.
14. Repa et al., Science 289:1524-1529, 2000.
15. Costet et al., J. Biol. Chem. 275:28240-28245, 2000.
16. Laffitte et al., Proc. Natl. Acad. Sci. USA 98:507-512, 2001.
17. Fazio et al., Proc. Natl. Acad. Sci. USA 94:4647-4652, 1997.
18. Linton et al., Science 267:1034-1037, 1995.
19. Bellosta et al., J. Clin. Invest. 96:2170-2179, 1995.
20. Tangirala, et al., J. Biol. Chem. 276:261-266, 2001.
21. Oram, Trends Cardiovasc. Med. 12:170-175, 2002.
22. Repa et al., Genes Dev. 14:2819-2830, 2000.
23. Yoshikawa et al., Mol. Cell. Biol. 21:2991-3000, 2001.
24. Joseph et al., J. Biol. Chem. 277:11019-11025, 2002.
25. Wang et al., Mol. Cell 3:543-553, 1999.
26. Kim et al., Mol. Cell Biol. 15:2582-2588, 1995.
27. Forman et al., Cell 81:687-693, 1995.
28. Forman et al., Proc. Natl. Acad. Sci. USA 94:4312-4317, 1997.
29. Kliewer, et al., Proc. Natl. Acad. Sci. USA 94:4318-4323, 1997.
30. Liang et al., J. Biol. Chem. 276:30987-30994, 2001.
31. Kennedy et al., J. Biol. Chem. 276:35008-35013, 2001.
32. Wang et al., Cell 77:53-62, 1994.
33. DeBose-Boyd et al., Proc. Natl. Acad. Sci. USA 98:1477-1482, 2001.
34. Lopez et al., Proc. Natl. Acad. Sci. USA 93:1049-1053, 1996.
35. Ou et al., Proc. Natl. Acad. Sci. USA 98:6027-6032, 2001.
36. Yoshikawa et al., J. Biol. Chem. 277:1705-1711, 2002.
37. Moore et al., J. Biol. Chem. 275:15122-15127, 2000.

1. A method of modulating LXR activity which comprises contacting said LXR with a compound of formula: 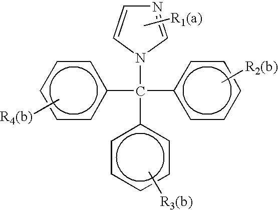
wherein (a) represents an integer from 0 to 3 and each (b) may be the same or different and represents an integer from 0 to 5;
wherein each R₁, R₂, R₃ and R₄ may be the same as or different from any other R₁, R₂, R₃ or R₄ and represents a moiety selected from the group consisting of C₁₋₄ alkyl, C₁₋₄ alkenyl, aryl, alkylaryl, halo, trihalomethyl, furanyl, thiophenyl, pyrrolyl, pyrazolyl, diazolyl, triazolyl, tetrazolyl, dithiolyl, oxathiolyl, oxazolyl, isoxazolyl, thiazolyl, isothiazolyl, oxadiazolyl, oxatriazolyl, dioxazolyl, isoxazinyl and piperazinyl, and
wherein the moiety may be unsubstituted or substituted with one or more substituent selected from the group consisting from methyl, ethyl, amino, halo, trihalomethyl and nitro.

2. A method of modulating lipid accumulation in a cell which comprises contacting said cell with a compound of formula: 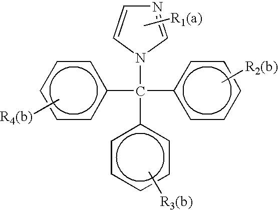
wherein (a) represents an integer from 0 to 3 and each (b) may be the same or different and represents an integer from 0 to 5;
wherein each R₁, R₂, R₃ and R₄ may be the same as or different from any other R₁, R₂, R₃ or R₄ and represents a moiety selected from the group consisting of C₁₋₄ alkyl, C₁₋₄ alkenyl, aryl, alkylaryl, halo, trihalomethyl, furanyl, thiophenyl, pyrrolyl, pyrazolyl, diazolyl, triazolyl, tetrazolyl, dithiolyl, oxathiolyl, oxazolyl, isoxazolyl, thiazolyl, isothiazolyl, oxadiazolyl, oxatriazolyl, dioxazolyl, isoxazinyl and piperazinyl, and
wherein the moiety may be unsubstituted or substituted with one or more substituent selected from the group consisting from methyl, ethyl, amino, halo, trihalomethyl and nitro.

3. A method of modulating lipid metabolism in a cell which comprises contacting said cell with a compound of formula: 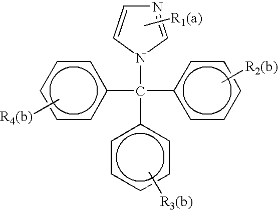
wherein (a) represents an integer from 0 to 3 and each (b) may be the same or different and represents an integer from 0 to 5;
wherein each R₁, R₂, R₃ and R₄ may be the same as or different from any other R₁, R₂, R₃ or R₄ and represents a moiety selected from the group consisting of C₁₋₄ alkyl, C₁₋₄ alkenyl, aryl, alkylaryl, halo, trihalomethyl, furanyl, thiophenyl, pyrrolyl, pyrazolyl, diazolyl, triazolyl, tetrazolyl, dithiolyl, oxathiolyl, oxazolyl, isoxazolyl, thiazolyl, isothiazolyl, oxadiazolyl, oxatriazolyl, dioxazolyl, isoxazinyl and piperazinyl, and
wherein the moiety may be unsubstituted or substituted with one or more substituent selected from the group consisting from methyl, ethyl, amino, halo, trihalomethyl and nitro.

4. A method of claim 1 wherein said compound is selected from the group consisting of Clotrimazole, T0901317, S883417 and S100250.

5. A method of claim 2 wherein said compound is selected from the group consisting of Clotrimazole, T0901317, S883417 and S100250.

6. A method of claim 3 wherein said compound is selected from the group consisting of Clotrimazole, T0901317, S883417 and S100250.

7. A method of modulating lipid metabolism in a cell which comprises contacting said cell with an LXR agonist.

8. A method of modulating lipid metabolism in a cell which comprises contacting said cell with an LXR antagonist.

9. A method of screening a test compound for specific LXR inhibitors which comprises:
(a) transfecting cells with one or more nucleic acids that encode
(1) the ligand-binding domain of an LXR,
(2) the ligand-binding domain of RXR,
(3) a reporter gene which is expressed upon activation of said LXR, and
(4) an internal control gene;
(b) incubating said cells with a test compound;
(c) assaying said cells for expression of said reporter gene and said control gene;
(d) normalizing the level of expression of said reporter gene to said control gene; and
(e) determining whether said reporter gene expression is decreased upon incubation with said test compound;
wherein decrease of said reporter gene expression indicates that the test compound is a specific LXR inhibitor.

10. A method of claim 9 wherein said LXR is selected from the group consisting of hLXRα and hLXRβ.

11. A method of claim 9 wherein said reporter gene is UAS_{GK}4 TK Luc.

12. A method of claim 9 wherein said control gene is CMX-β-gal.
